(19) United States
(12) Patent Application Publication
(10) Pub. No.: US 2023/0310870 A1
(43) Pub. Date: Oct. 5, 2023

(54) Rechargeable Neuromodulation Device

(71) Applicant: Saluda Medical Pty Ltd, Level 1 (AU)

(72) Inventors: Peter Scott Vallack Single, Artarmon (AU); Dean Michael Karantonis, Artarmon (AU)

(73) Assignee: Saluda Medical Pty Ltd, Level 1 (AU)

(21) Appl. No.: 18/041,679

(22) PCT Filed: Aug. 13, 2021
(86) PCT No.: PCT/AU2021/050895

§ 371 Date: Feb. 14, 2023

(30) Foreign Application Priority Data
Aug. 14, 2020 (AU) 2020902899

(51) Int. Cl. A61N 1/378; A61N 1/372; A61N 1/36

(52) U.S. Cl. CPC A61N 1/3787; A61N 1/37223; A61N 1/36135; A61N 1/36146

(57) ABSTRACT

An implantable pulse generator device (110) comprising a processor (117) configured to: receive, in a charging mode, electromagnetic radiation (106) from a charging device (102) wherein the electromagnetic radiation (106) transfers energy to the implantable device (110) to charge an energy storage device (104); measure, in a measurement mode, an electrical field parameter signal representing a neural response; and selectively transition between the charging mode and the measurement mode, such that the implantable device (110) does not receive electromagnetic radiation (106) from the charging device (102) during the measurement of the electrical field parameter signal.

### CROSS-REFERENCE TO RELATED APPLICATIONS

The present application is a U.S. national stage patent application of PCT Patent Application No. PCT/AU2021/050895 filed on Aug. 13, 2021, which claims priority from Australian Provisional Patent Application No. 2020902899 filed on 14 Aug. 2020, the contents of which are incorporated herein by reference in their entirety.

### TECHNICAL FIELD

The disclosure relates to a rechargeable, implantable neuromodulation device and in particular to neuromodulation devices that are configured to measure neurological activity.

### BACKGROUND

There are a range of situations in which it is desirable to apply neural stimuli in order to give rise to a compound action potential (CAP). For example, neuromodulation is used to treat a variety of disorders including chronic pain, Parkinson's disease, and migraine. A neuromodulation system applies an electrical pulse to tissue in order to generate a therapeutic effect. When used to relieve chronic pain, the electrical pulse is applied to the dorsal column (DC) of the spinal cord. Such a system typically comprises an implanted electrical pulse generator, and a power source such as a battery that may be rechargeable by transcutaneous inductive transfer. An electrode array is connected to the pulse generator, and is positioned in the dorsal epidural space above the dorsal column. The electrode array applies an electrical pulse to the dorsal column, which causes the depolarisation of neurons, and generation of propagating action potentials. This stimulates the nerve fibres and as a result, inhibits the transmission of pain from that segment in the spinal cord to the brain. The electrode array applies stimuli continuously to sustain the pain relief effects.

While the clinical effect of spinal cord stimulation (SCS) is well established, the precise mechanisms involved are poorly understood. The DC is the target of the electrical stimulation, as it contains the afferent AP fibres of interest. AP fibres mediate sensations of touch, vibration and pressure from the skin, and are thickly myelinated mechanoreceptors that respond to non-noxious stimuli. The prevailing view is that SCS stimulates only a small number of Aβ fibres in the DC. The pain relief mechanisms of SCS are thought to include evoked antidromic activity of AP fibres having an inhibitory effect, and evoked orthodromic activity of Aβ fibres playing a role in pain suppression. It is also thought that SCS recruits Aβ nerve fibres primarily in the DC, with antidromic propagation of the evoked response from the DC into the dorsal horn thought to synapse to wide dynamic range neurons in an inhibitory manner.

Neuromodulation may also be used to stimulate efferent fibres, for example to induce motor functions. In general, the electrical stimulus generated in a neuromodulation system triggers a neural action potential which then has either an inhibitory or excitatory effect. Inhibitory effects can be used to modulate an undesired process such as the transmission of pain, or to cause a desired effect such as the contraction of a muscle.

The action potentials generated among a large number of fibres sum to form a compound action potential (CAP). The CAP is the sum of responses from a large number of single fibre action potentials. The CAP recorded is the result of a large number of different fibres depolarising. The propagation velocity is determined largely by the fibre diameter and for large myelinated fibres as found in the dorsal root entry zone (DREZ) and nearby dorsal column the velocity can be over 60 ms⁻¹. The CAP generated from the firing of a group of similar fibres is measured as a positive peak potential P1, then a negative peak N1, followed by a second positive peak P2. This is caused by the region of activation passing the recording electrode as the action potentials propagate along the individual fibres.

For effective and comfortable operation, it is useful to maintain induced stimuli amplitude or delivered charge above a recruitment threshold, below which an induced stimulus may fail to recruit any neural response. It is also useful to induce stimuli which are below a comfort threshold, above which uncomfortable or painful percepts arise due to increasing recruitment of Aδ fibres which are thinly myelinated sensory nerve fibres associated with acute pain, cold and pressure sensation. In almost all neuromodulation applications, a single class of fibre response is desired, but the stimulus waveforms employed can recruit other classes of fibres which cause unwanted side effects, such as muscle contraction if motor fibres are recruited. The task of maintaining appropriate stimulus amplitude is made more difficult by electrode migration and/or postural changes of the implant recipient, either of which can significantly alter the neural recruitment arising from a given stimulus, depending on whether the stimulus is applied before or after the change in electrode position or user posture. Postural changes alone can cause a comfortable and effective stimulus regime to become either ineffectual or painful.

Typically, the stimuli can be delivered within a therapeutic range (above the recruitment threshold and below the comfort threshold) by adjusting the amplitude of applied stimulus based on a feedback signal. The feedback signal is based on a measured CAP signal, detected by an electrode connected to the nerve fibres upstream of the stimulating electrode. Based on the CAP signal, the amplitude of the applied stimulus can be adjusted to maintain the nerve stimulus within the therapeutic range. A method for achieving this is disclosed in U.S. Pat. Nos. 9,381,356 B2, and 10,500,399 B2 the contents of which is hereby incorporated.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising, will be understood to imply the inclusion of a stated element, integer or step, or group of elements, integers or steps, but not the exclusion of any other element, integer or step, or group of elements, integers or steps.

### SUMMARY

There is provided an implantable pulse generator device comprising a processor configured to:
(i) receive, in a charging mode, electromagnetic radiation from a charging device wherein the electromagnetic radiation transfers energy to the implantable device to charge an energy storage device;
(ii) measure, in a measurement mode, an electrical field parameter signal representing a neural response; and
(iii) selectively transition between the charging mode and the measurement mode, such that the implantable device does not receive electromagnetic radiation from the charging device during the measurement of the electrical field parameter signal.

In some embodiments, the processor is further configured to apply a neural stimulation signal to a neural pathway in at least one of: the charging mode; and the measurement mode.

In some embodiments, the processor is further configured to signal the charging device to cease transmission of the electromagnetic radiation before the measurement of the electrical field parameter signal.

In some embodiments, the signal to the charging device to cease transmission of the electromagnetic radiation is a reflected impedance indicative of a charged energy storage device.

In some embodiments, the reflected impedance is maintained for a stimulation-recording period during which a neural stimulus signal is applied and a corresponding electrical field parameter signal is measured.

In some embodiments, the signal to the charging device to cease transmission of the electromagnetic radiation is a wireless electromagnetic radio frequency signal.

In some embodiments, the radio frequency signal is within a medical implant communication service (MICS) band.

In some embodiments, the signal to the charging device to cease transmission of the electromagnetic radiation is maintained while in the measurement mode.

In some embodiments, the processor periodically operates in the measurement mode.

In some embodiments, the processor is configured to perform an adhoc transition to the measurement mode at random instances including one or more of: a time when the charger begins actively transferring electromagnetic radiation; and a time when the implantable device is directed to measure the electrical field parameter signal.

In some embodiments, the measurement of the electrical field parameter signal may include the measurement of at least one of: an ECAP; a non-evoked CAP; a local field potential (LFP); a slow response; a physiological parameter; or a like neural response parameter.

There is further provided a method performed by a processor of an implantable pulse generator device to charge an energy storage device, the method comprising:
(i) receiving, in a charging mode, electromagnetic radiation from a charging device wherein the electromagnetic radiation transfers energy to the implantable device to charge an energy storage device;
(ii) measuring, in a measurement mode, an electrical field parameter signal representing a neural response; and
(iii) selectively transitioning between the charging mode and the measurement mode, such that the implantable device does not receive electromagnetic radiation from the charging device during the measurement of the electrical field parameter signal.

There is further provided a system comprising:
an implantable pulse generator device comprising a processor configured to:
   (i) receive, in a charging mode, electromagnetic radiation from a charging device wherein the electromagnetic radiation transfers energy to the implantable device to charge an energy storage device;
   (ii) measure, in a measurement mode, an electrical field parameter signal representing a neural response; and
      (iii) selectively transition between the charging mode and the measurement mode, such that the implantable device does not receive electromagnetic radiation from the charging device during the measurement of the electrical field parameter signal; and
a charger configured to transition from an active state to a standby state in response to receiving a signal from the implantable device, wherein in the active state the charger transmits electromagnetic radiation to the energy storage device and wherein in the standby state the charger does not transmit electromagnetic radiation to the energy storage device.

There is further provided a non-transitory computer readable medium configured to store software instructions that when executed cause a processor to perform the above method.

There is further provided a charger comprising a processor configured to transition from an active state to a standby state according to a predetermined duty cycle at a predetermined frequency, wherein in the active state the charger transmits electromagnetic radiation to an implantable device and wherein in the standby state the charger does not transmit electromagnetic radiation to the implantable device.

In some embodiments, the charger is further configured to: receive an interrupt signal from the implantable device; and in response to the received interrupt signal, perform at least one of: ceasing the transmission of electromagnetic radiation to the implantable device; and commencing the transmission of electromagnetic radiation to the implantable device.

In some embodiments, the predetermined duty cycle is configurable based on a received configuration signal.

In some embodiments, the configuration signal is received from the implantable device.

There is further provided an implantable pulse generator device comprising a processor configured to selectively operate in one of a charging mode and a measurement mode, wherein the processor is further configured to:
detect electromagnetic radiation from a charging device wherein the electromagnetic radiation transfers energy to the implantable device to charge an energy storage device and wherein the electromagnetic radiation is transferred at a predetermined frequency and duty cycle to transfer energy during a first part of a charge cycle and to stop transferring energy during a second part of the charge cycle;
receive, in the charging mode, electromagnetic radiation from the charging device during the first part of the charge cycle;
apply a neural stimulus to a neural pathway; and
a measure, in the measurement mode, an electrical field parameter signal during the second part of the charge cycle.

In some embodiments, the processor is further configured to:
determine the predetermined frequency and duty cycle from the electromagnetic radiation; and
perform stimulation cycles at the predetermined frequency to synchronise the stimulation cycles with the second part of the charge cycle, wherein each stimulation cycle comprises applying a neural stimulus to the neural pathway, and measuring, in the measurement mode, the electrical field parameter signal.

In some embodiments, the processor of the implantable pulse generator device is further configured to:
detect the presence of an emitting device within a charging distance of the implantable device, the emitting device transmitting electromagnetic radiation to the implantable device;
verify whether the detected emitting device is the charging device; and
in response to a positive verification of the emitting device as the charging device, cause the implantable device to transition, from any other operational routine, to a smart charging operational routine in which the processor executes steps (i) to (iii).

In some embodiments, the processor is further configured to: detect the presence of the emitting device by processing a detection signal received from a sensor associated with a charging coil of the implantable device.

In some embodiments, the processor is further configured to: detect the presence of the emitting device by processing a detection signal received from an amplifier component of the implantable device, wherein the amplifier component may be configured to measure a noise signal associated with the electromagnetic radiation received from the emitting device.

In some embodiments, the verifying of the emitting device as the charging device includes processing the noise signal to recognize a noise signature associated with the charging device.

In some embodiments, the processor is further configured to: transmit one or more interrupt signals to the emitting device; and detect a response of the emitting device to the one or more interrupt signals, to verify whether the emitting device is the charging device.

In some embodiments, the response of the emitting device that enables the processor to positively verify the emitting device as the charging device is an acknowledgment response involving the ceasing of the transmission of the electromagnetic radiation by the emitting device.

In some embodiments, the processor is further configured to:
detect an absence of the acknowledgment response of the emitting device within a first pre-determined time period; and
in response to the absence of the acknowledgment response within the first pre-determined time period, cause the implantable device to transition, from any other operational routine, to a static stimulus operational routine in which the processor does not execute steps (i) to (iii), and instead executes the step of:
applying a neural stimulus without a corresponding measurement of an electrical field parameter signal.

In some embodiments, the processor is further configured to:
detect the presence of the acknowledgment response of the emitting device within a second pre-determined time period occurring after the first time period; and
in response to the detection of the acknowledgment response in the second time period, positively verify the emitting device as the charging device, to cause the implantable device to transition from the static stimulus operational routine to the smart charging operational routine.

In some embodiments, the method to charge an energy storage device further comprises:
detecting the presence of an emitting device within a charging distance of the implantable device, the emitting device transmitting electromagnetic radiation to the implantable device;
verifying whether the detected emitting device is the charging device; and
in response to a positive verification of the emitting device as the charging device, causing the implantable device to transition, from any other operational routine, to a smart charging operational routine in which the processor executes steps (i) to (iii).

In some embodiments, the method further comprises: transmitting one or more interrupt signals to the emitting device; detecting a response of the emitting device to the one or more interrupt signals; and verifying, based on the response of the emitting device, whether the emitting device is the charging device.

In some embodiments, the method further comprises:
detecting an absence of an acknowledgment response of the emitting device within a first pre-determined time period, wherein the acknowledgment response involves the ceasing of the transmission of the electromagnetic radiation by the emitting device; and
in response to the absence of the acknowledgment response within the first pre-determined time period, causing the implantable device to transition, from any other operational routine, to an open loop operational routine in which the processor does not execute steps (i) to (iii), and instead executes the step of:
applying a neural stimulus without a corresponding measurement of an electrical field signal.

In some embodiments, the method further comprises:
detecting the presence of the acknowledgment response of the emitting device within a second pre-determined time period occurring after the first time period; and
in response to the detection of the acknowledgment response in the second time period, positively verifying the emitting device as the charging device, to cause the implantable device to transition from the static stimulus operational routine to the smart charging operational routine.

### BRIEF DESCRIPTION OF DRAWINGS

FIG. 1 illustrates an implanted neuromodulation device and charger;

FIG. 2 is a schematic illustration of a neuromodulation device;

FIG. 3 is a flowchart of a method performed by the device of FIG. 2;

FIG. 4 illustrates exemplary timing sequences according to the method of FIG. 3;

FIG. 5 is a schematic illustration of a charging device;

FIG. 6 is a flowchart of a method performed by the device of FIG. 5;

FIG. 7 is a flowchart of a method performed by the device of FIG. 2;

FIG. 8 is a schematic illustration of a neuromodulation device;

FIG. 9 is a schematic illustration of a neuromodulation device; and

FIG. 10 is a flowchart of a method performed by the device of FIG. 2.

### DESCRIPTION OF EMBODIMENTS

As mentioned above, it is useful to maintain stimuli amplitude within a therapeutic range to maintain effective and comfortable neural stimulation. That is, the stimuli are above a recruitment threshold and below a comfort threshold. A neural modulation device can adjust the amplitude of applied stimulus based on the measurement of a compound action potential (CAP) signal that is evoked in response to the stimulus (referred to as an “ECAP signal”) to keep the stimuli within this therapeutic range. A neural modulation device operating in this manner is said to be operating in closed loop mode (i.e., with reference to the use of the ECAP as a type of feedback signal). This may also be referred to as a closed loop neural stimulus (CLNS). An ECAP signal typically has a maximum amplitude in the range of microvolts, whereas an applied stimulus signal evoking the CAP is typically several volts.

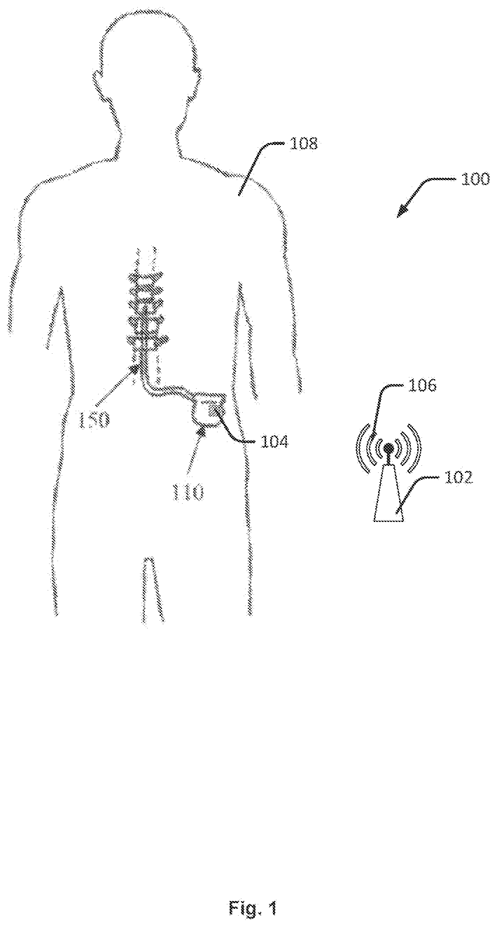

Implantable devices that perform CLNS are battery powered and use intermittent charging of the battery to remain operational. FIG. 1 illustrates a wireless charging system 100 to charge a battery in an implanted neuromodulation device. Wireless charging system 100 comprises a charging device (or “charger”) 102 to transfer energy to an energy storage device 104 (e.g., a battery) of the implanted device 110 using electromagnetic radiation 106. The electromagnetic radiation induces a charging current in the implanted device 110 which can be used to charge the energy storage device 104. Implanted device 110 is implanted at a suitable location in a patient 108, such as the lower abdominal area or posterior superior gluteal region.

Charger 102 detects the charge level of the energy storage device 104 as a reflected impedance. Using this reflected impedance, in response to the charge level of the energy storage device 104 reaching a maximum, the charger 102 detects that the energy storage device is charged, and automatically ceases transmitting electromagnetic radiation 106.

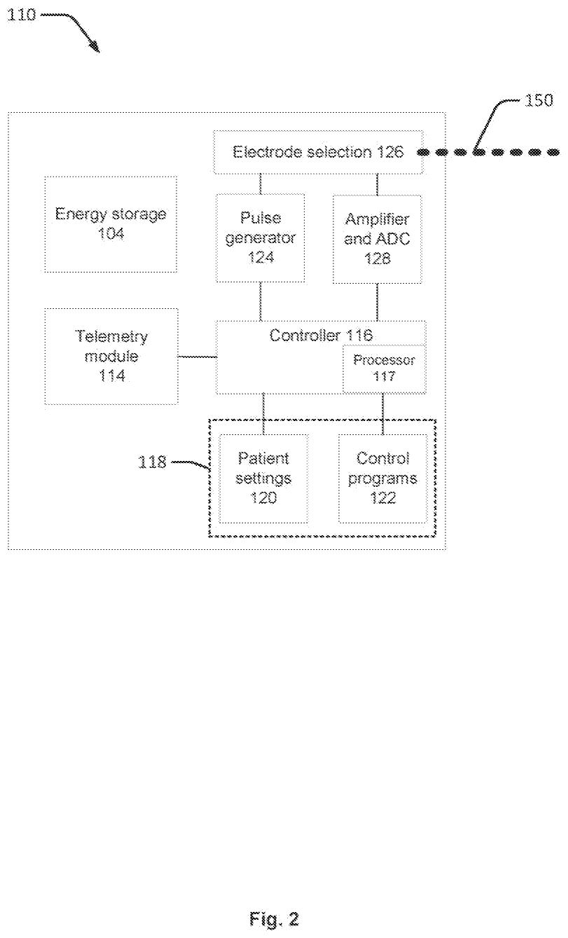

FIG. 2 is a block diagram of implanted device 110 which comprises an energy storage device 104 and a telemetry module 114. Energy storage device 104 may be any suitable energy storage device, such as a battery or capacitor. Telemetry module 114 transfers power and/or data between an external device and other modules of device 110. For example, telemetry module 114 may receive power from charger 102 to energy storage device 104. Telemetry module 114 may utilise any suitable type of transcutaneous communication such as infrared (IR) and other electromagnetic radiation, including capacitive and inductive transfer.

Module controller 116 has an associated memory 118 storing patient settings 120, control programs 122 and the like. Controller 116 controls a pulse generator module 124 to generate stimuli in the form of current pulses in accordance with the patient settings 120 and control programs 122. Electrode selection module 126 switches the generated pulses to the appropriate electrode(s) of electrode array 150, for delivery of the current pulse to the tissue surrounding the selected electrode. Measurement circuitry 128 is configured to capture measurements of neural responses sensed at sense electrode(s) of the electrode array as selected by electrode selection module 126.

The neural response is determined by the measurement of an electrical field parameter signal by the measurement circuitry 128 components. For example, the measurement of the electrical field parameter signal may include the measurement of at least one of: an evoked neural compound action potential (ECAP); a non-evoked neural compound action potential (nECAP); a local field potential (LFP); a slow response, or a physiological parameter (such as EMG, ECoG, and EKG). Although the embodiments described herein relate to the measurement of an ECAP signal, the skilled addressee will appreciate that measurement of any other type of electrical field parameter indicating a neural response may be performed alternatively, or in addition.

The desired magnitude of the charging potential depends on the type of battery and is typically in the order of one to five volts. This causes an induced potential in the measurement electrodes, which can be several orders of magnitude greater than the ECAP signal (microvolts) preventing detection, or accurate measurement, of the ECAP signal. As a result, some neuromodulation devices cannot operate to alter the applied neural stimulus based on a measurement of the ECAP (i.e., closed loop operation) while the battery is charging. Instead, the devices are limited to “open loop” control, where the amplitude used for the applied stimulus is not based on a consideration of a dynamically measured ECAP signal, which may result in induced stimuli falling outside of the therapeutic range leading to unwanted effects such as discomfort for the patient.

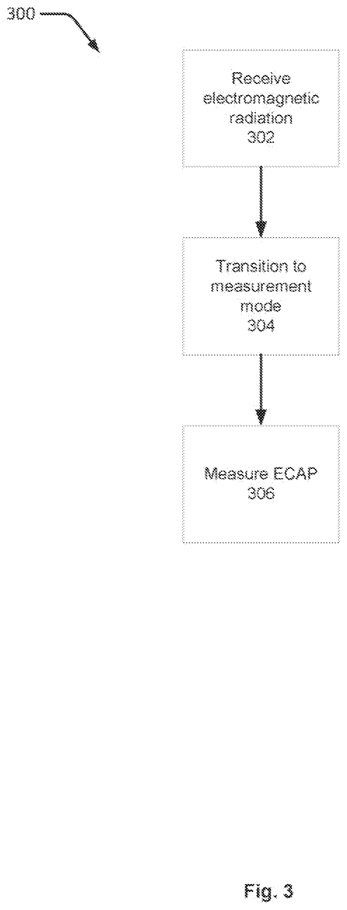

In order to address this problem, this disclosure provides an implantable neural stimulation device, which is also referred to as an “implantable pulse generator” (IPG) device, 110 that operates to control the measurement of an evoked neural compound action potential (ECAP) signal in response to the charging of the device 110. In the case that the ECAP signal is produced in response to the application of a neural stimulus to a neural pathway by the implantable device 110, this enables the device 110 to perform CLNS while being charged by a charging device (e.g., the charger 102). The implantable neural stimulus device 110 comprises a controller 116 having a processor 117 configured to perform method 300 of FIG. 3. When performing method 300, processor 117 selectively operates in one of a charging mode and a measurement mode.

At step 302, the processor 117 is in the charging mode and is configured to receive electromagnetic radiation 106 from the charger 102. Electromagnetic radiation 106 transfers energy to implantable device 110 to charge the energy storage device 104 as described above.

Processor 117 directs the application of a neural stimulus signal to a neural pathway. Specifically, implantable device 110 generates and applies a neural stimulus signal to the neural pathway though electrodes 150. The applied stimulus signal evokes a compound action potential (ECAP) signal response in the neural pathway which is measured at a point along the neural pathway from where the stimulus signal was applied. This ECAP signal may be measured and used as a feedback signal to adjust the amplitude of the neural stimulus signal.

Before measuring the ECAP signal corresponding to the application of a neural stimulus signal, processor 117 selectively transitions from the charging mode and to the measurement mode such that the implantable device 110 does not receive electromagnetic radiation 106 from the charger 102 during the measurement of the ECAP. In one embodiment, the measurement mode involves the processor 117 signaling to the charging device 102 to cease transmission of electromagnetic radiation 106. As a result, charging device 102 transmits no electromagnetic radiation 106 during the period when implantable device 110 measures the ECAP signal. The ECAP signal is then detected and measured at a point along the neural pathway at step 306. This enables the ECAP signal to be used as a feedback signal to adjust the neural stimulus signal (i.e., to perform CLNS). Charging device 102 can resume transmission of electromagnetic radiation 106 after measurement of the ECAP. In this case the processor 117 selectively transitions back to the charging mode to enable the charging of the energy storage device 104.

In some embodiments, implantable device 110 signals to the charging device 102 to cease transmission of the electromagnetic radiation before generating and applying the neural stimulus signal. That is, the processor 117 transitions to the measurement mode prior to applying the neural stimulus signal. In such embodiments, implantable device 110 comprises a charging controller and a stimulation module. The stimulation module has an ‘enable’ input that is connected to the charging controller. The charging controller clears the ‘enable’ signal to disable stimulation and then signals to the charging device 102 to transmit electromagnetic radiation. After a period of time, such as the calculated time between stimulation pulses, the charging controller signals to charging device 102 to cease transmission of the electromagnetic radiation and sets the ‘enable’ signal to enable stimulation.

In some implementations, after signalling to the charging device 102 to cease transmission, implantable device 110 waits for an acknowledgement from the charging device 102 or detects the actual ceasing of the electromagnetic radiation before enabling stimulation and subsequent measurement of the ECAP signal.

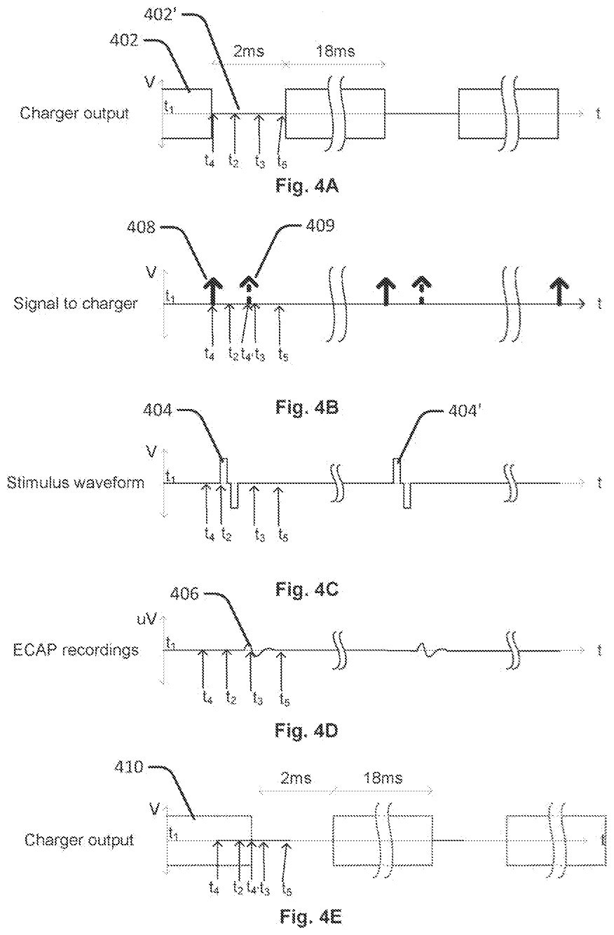

The timing sequence of these processes are shown in FIGS. 4A to 4E. FIG. 4A is the output of charger 102, while FIGS. 4B, C and D take place in device 110.

At an initial time t₁, charger 102 is transmitting electromagnetic radiation 106 as indicated by transmission signal 402. Transmission signal 402 is illustrated as a rectangular section because the electromagnetic radiation is typically a high frequency alternating current (AC) signal with a frequency typically between 250 KHz and 400 KHz, though frequencies as high as 5 MHz have been used. At this time, processor 117 is operating in charging mode and performing step 302 of method 300. At a later time, t₂, processor 117 applies a stimulus signal 404 and measures the evoked CAP signal 406 at a later time, t₃. As discussed above, CAP signal 406 is used as feedback to adjust the amplitude of future stimulus waveforms 404′. At a time t₄, which is prior to t₃, processor 117 transmits an interrupt signal 408 to charger 102 to cease transmission of electromagnetic radiation as indicated by transmission voltage dropping to zero at 402′ (i.e., during the measurement mode).

FIGS. 4C and 4D illustrate stimulus signal 404 and ECAP signal 406 as having no temporal overlap. However, in practice, stimulus signal 404 and ECAP signal 406 may overlap depending on the physical arrangement of the electrodes and propagation velocity of stimulus signal 404 along the nerve fibres. In some embodiments, the ECAP signal 406 is measured at the same time that stimulus signal 404 is applied. That is, t₂ and t₃ occur at the same time.

As charger 102 is not transmitting electromagnetic radiation at time t₃ when ECAP signal 406 is being measured, implantable device 110 can detect and accurately measure ECAP signal 406 to use it as a feedback signal.

FIGS. 4A to 4D illustrate an implementation of method 300 when interrupt signal 408 is transmitted not only before ECAP signal 406 is measured, but also before stimulus signal 404 is applied to the neural pathway. That is, t₄ is prior to t₃ and t₂.

At some later time, t₅, charger 102 recommences transmission of electromagnetic radiation 106 to continue charging of the energy storage device 104. In some embodiments, processor 117 transmits a recommence signal (not shown) after the ECAP signal 406 has been measured, causing charger 102 to recommence transmission of electromagnetic radiation 106.

In other embodiments, the neural stimulus signal is applied during the transmission of the electromagnetic radiation (i.e., during the charging mode) and the processor 117 transitions from the charging mode to the measurement mode after the application of the stimulus signal, and before the measuring of the corresponding ECAP signal. As before, charging device 102 only recommences transmission of electromagnetic radiation 106 after device 110 has measured the ECAP signal. This is illustrated by the interrupt signal 409 of the implantable device 110 shown in FIG. 4B, and the transmission signal output 410 of the charger 102 shown in FIG. 4E (both signals being depicted in dashed lines). In this embodiment, the processor 117 transmits the interrupt signal 409 to charger 102 at a time t_{4′} (shown only in FIGS. 4B and 4E) to cease transmission of electromagnetic radiation 106 according to the transmission signal 410. Time t_{4′} occurs after time t₂, which is when the processor 117 applies the stimulus signal 404, but prior to time t₃ when the processor 117 measures the evoked CAP signal 406.

In some embodiments, processor 117 continuously transmits interrupt signal 408 for a period of time required to both apply stimulus signal 404 and measure corresponding ECAP signal 406. This period is referred to as a stimulation-recording period. At the end of the stimulation-recording period, processor 117 ceases transmission of interrupt signal 408 causing charger 102 to recommence transmission of electromagnetic radiation 106. In the example illustrated in FIGS. 4A to 4E, the stimulation-recording period is two milliseconds.

In some embodiments, charger 102 automatically recommences transmission of electromagnetic radiation 106 after a predetermined period from receiving the first interrupt signal that signals the ceasing of the transmission. The predetermined time period may be stored on charger 102 or communicated to charger 102 from implantable device 110, either through interrupt signal 408 or through a separate communication.

It will be appreciated that interrupt signal 408 can take many forms. In one example, interrupt signal 408 is a reflected impedance which is indicative of a charged energy storage device. That is, processor 117 simulates an electrical load on telemetry module 114 which is equivalent to the electrical load that would be experienced if the energy storage device 104 were fully charged. This reflected impedance is detected by charger 102, causing charger 102 to cease transmission of electromagnetic radiation 106.

An advantage of using the reflected impedance as interrupt signal 408 is that there would be no requirement to modify charger 102 if such a charger were already in use.

In another example, interrupt signal 408 is a wireless electromagnetic radio frequency signal. For example it may be a radio frequency signal within a medical implant communication service (MICS) band.

Processor 117 of device 110 may periodically operate in the measurement mode to provide a prolonged therapeutic effect to patient 108. In the example illustrated in FIGS. 4A to 4D, processor 117 periodically alternates between charging mode and measurement mode with a period of twenty milliseconds (which corresponds to a frequency of fifty hertz). In this case, processor 117 operates in measurement mode for a stimulation-recording period of two milliseconds and then operates in charging mode for the remainder of the twenty millisecond period (i.e. eighteen milliseconds). There is no overlap between the measurement mode and the charging mode.

In other embodiments, the processor 117 may perform one or more adhoc transitions to the measurement mode. These transitions may occur at random instances, such as, for example, whenever the charger 102 begins actively transferring electromagnetic radiation 106 to the implantable device 110. In some embodiments, the 117 transitions may occur when the implantable device 110 is directed to measure an electrical parameter representing a neural response, such as an ECAP, a non-evoked CAP, a local field potential (LFP), a slow response, a physiological parameter (such as EMG, ECoG, and EKG), or a like parameter.

In some implementations, processor 117 monitors a charge level of the energy storage device 104. While the charge level is above a predetermined threshold, processor 117 continuously generates interrupt signal 408. In this case, if device 110 comes into operational range of a charger 102, the charger will not transmit electromagnetic radiation as it will detect interrupt signal 408. When the charge level drops below the predetermined threshold, processor 117 will execute method 300 as described above, enabling the energy storage device 104 to be charged by the charger 102 while also enabling the adjustment of the stimulus signal based on a measured ECAP signal.

In some implementations, processor 117 executes method 300 continuously enabling the energy storage device 104 to charge each time device 110 moves within operational range of charger 102.

The instructions for method 300 are stored in control programs 122 and are embodied in a software program written in a programming language such as C++ or Java. The resulting source code is then compiled and stored as computer executable instructions.

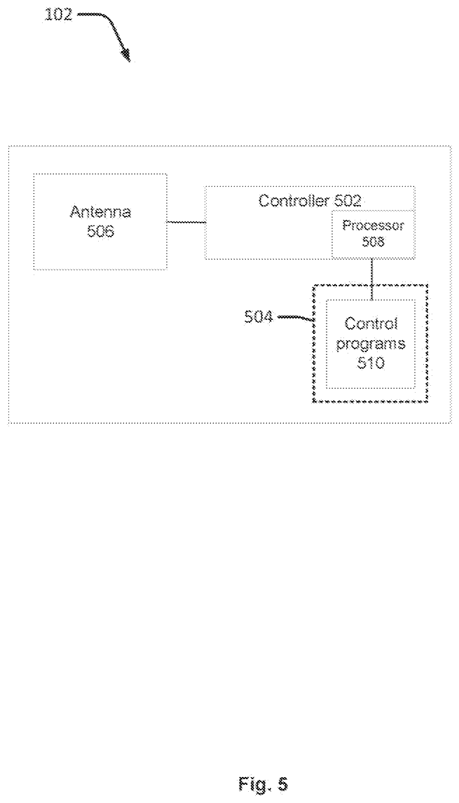

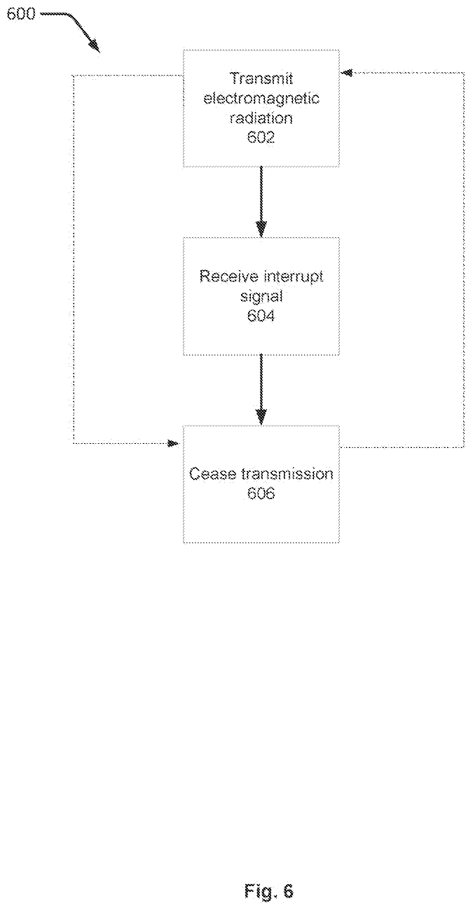

FIG. 5 is a schematic illustration of charging device 102 which comprises a controller 502, with associated memory 504 and an antenna 506. Controller 502 comprises processor 508 for executing instructions stored in charging control program 510 on memory 504. Antenna 506 is configured to transmit electromagnetic radiation 106 to implantable device 110. Charging control program 510 comprises instructions to perform method 600 which is illustrated in FIG. 6.

At step 602, controller 502 controls charging device 102 to transmit electromagnetic radiation to implantable device 110. As mentioned above, charger 102 transfers energy to the energy storage device 104. An interrupt signal 408 is received at step 604 causing controller 502 to execute step 606 by ceasing transmission of electromagnetic radiation 102.

In some embodiments, method 600 is executed in a loop such that step 602 is executed again after step 606.

In some embodiments, method 600 loops back to step 602 when interrupt signal 408 is no longer received. That is, controller 502 executes step 606 while interrupt signal 408 is received and when interrupt signal 408 is not received it executes step 602.

In some embodiments, method 600 loops back to step 602 after a predetermined period of time of ceasing transmission. The predetermined period of time may be stored in charging control program 510. Alternatively, the predetermined period of time may be derivable from interrupt signal 408 or received as a separate communication from implantable device 110.

In some embodiments, method 600 loops back to step 602 when a separate recommence signal (not shown) is received.

In some embodiments, charging device 102 is configured to transmit electrical energy (i.e., electromagnetic radiation 106) according to a pre-determined duty cycle and predetermined frequency. That is, charger 102 is configured to transition from an active state in a first part of a charging cycle to a standby state in a second part of a charging cycle according to a predetermined duty cycle. In the active state, charger 102 transmits electromagnetic radiation to the implantable device 110 and in the standby state charger 102 does not transmit electromagnetic radiation to the implantable device. The duty cycle is defined by a relationship between the temporal duration of the first part of the charging cycle and the temporal duration of the second part of the charging cycle. The first and second parts of the charging cycle are repeated at the predetermined frequency.

In some implementations, the charger 102 performs the transmission of electrical energy according to the pre-determined duty cycle and frequency without receiving an interrupt signal. That is, with reference to method 600, the charger 102 may be configured to transition back and forth between the active state (at step 602), and the standby state (at step 606), without receiving an interrupt signal from the implantable device 110. Direct transition of the charger 102 from step 602 to 606, and the corresponding reverse transition, are shown as dashed lines in FIG. 6.

In some embodiments, the charger 102 is configured to initiate the transmission of electrical energy according to the pre-determined duty cycle and frequency automatically on the detection of the implantable device 110. The charger 102 includes detection circuitry configured to enable the detection of the implantable device 110. For example, the charger 102 may sense the presence of the implantable device 110 via a change in the reflective impedance detected within a coil, or similar component, of the charger 102 and initiate charging according to the pre-determined duty cycle.

Although, the charger 102 is not required to receive an interrupt signal to commence transmission of electrical energy according to the pre-determined duty cycle and frequency, the charger 102 may still receive such an interrupt signal. In some implementations, the charger 102 is configured to respond to the received interrupt signal to alter the transmission of electrical energy in a pre-specified manner. For example, the charger 102 may be configured to respond by: ceasing transmission of the electrical energy entirely (e.g., invoking a turn-off” function); commencing a pre-determined cool-off period in which transmission of electrical energy is immediately ceased and then resumed on termination of the cool-off period according to the pre-determined duty cycle; advancing to the second part of the current charging cycle; and/or resetting the duty cycle. For the purposes of discussion, when operating in this embodiment the charger 102 will be referred to as the master charger 102.

In some implementations, the predetermined frequency and duty cycle is configurable based on a received configuration signal. For example, the duty cycle and frequency of the charger may be transmitted to it from the implantable device 110, or from a remote control used by the patient to control the implantable device, or, the frequency and duty cycle may be established in a clinic by a clinician.

In some embodiments, implantable device 110 is configured to receive electromagnetic radiation from charger 102 discussed above. Processor 117 selectively operates in one of a charging mode and a measurement mode based on whether the charger 102 is in the first part of the charging cycle or the second part of the charging cycle. Processor 117 detects the first and second parts of the charging cycle in response to receiving electromagnetic radiation and operates in the charging mode during the first part of the charging cycle. As before, when in the charging mode, electromagnetic radiation is received from charging device 102 to charge the energy storage device 104.

In some embodiments, processor 117 determines the predetermined frequency and duty cycle of master charger 102 by monitoring the electromagnetic radiation from master charger 102. In one embodiment, processor 117 measures the time between successive first parts of the charge cycle to determine the predetermined frequency (being the inverse of the measured time period) and measures the duration of the first part of the charge cycle to determine the predetermined duty cycle.

Processor 117 can be configured to periodically perform stimulation cycles at the predetermined frequency such that the stimulation cycles are synchronised with the second part of the charge cycle. In each stimulation cycle, processor 117 applies a neural stimulus to the neural pathway and measures a ECAP response signal. The result of this is that processor 117 performs a stimulation cycle each time master charger 102 ceases transmission of electromagnetic radiation.

In some embodiments, processor 117 does not perform a complete stimulation cycle during each second part of the charge cycle. Rather, processor 117 is configured to perform an incomplete stimulation cycle by only applying a neural stimulus during the second part of the charge cycle and not measuring an evoked neural action potential. Processor 117 may periodically perform complete stimulation cycles to adjust the level of stimulation. That is, processor 117 may perform incomplete stimulation cycles interspersed with complete stimulation cycles. As a result of processor 117 only occasionally performing a complete stimulation cycle, the energy storage device 104 is able to charge faster as processor 117 consumes less energy when performing incomplete stimulation cycles.

Using the detected electromagnetic radiation, processor 117 is able to predict when master charger 102 will be in the second part of the charge cycle as the second part of the charge cycle is also repeated at the predetermined frequency. Synchronising the stimulation cycles is then a matter of processor 117 performing the stimulation cycles at the predetermined frequency and with a phase off-set that allows the stimulation cycles to fall within the second part of the charge cycle. The phase off-set will be determined by the predetermined duty cycle and is effectively a timing off-set.

For example, processor 117 determines a start of each coming charge cycle using the detected first part of the charge cycle and the measured time between successive starts. A phase delay, or timing off-set, equal to the duration of the first part of the charge cycle is then be added to these times to yield predicted times for the second parts of the charge cycle.

In some embodiments, the phase delay is only calculated once and the stimulation cycles are then performed at the predetermined frequency. In other embodiments, the phase off-set is periodically determined to ensure that the stimulation cycles remain synchronised with the second part of the charge cycle.

In some embodiments, the measurements of the ECAP are used to determine the upper and lower stimulation thresholds rather than in a feedback loop to maintain the stimulus between the respective threshold levels, as for the embodiments described above. Determining the relevant thresholds enables the delivery of stimulation at an intensity that does not result in the detection of an ECAP response signal.

For example, the ECAP measurements may be used to determine the minimum level of stimulation required to evoke a detectable response (i.e., the ECAP may be recorded for a level of stimulation, and the implanted device configured to reduce the stimulation level until the ECAP is no longer detectable). Applying stimulation at an intensity below this value can be considered as subthreshold stimulation. Subthreshold stimulation may be performed without measuring a corresponding evoked compound action potential. Rather, in this embodiment, the processor 117 is configured to only apply a neural stimulus to the neural pathway which may occur when the implantable device 110 is being charged by the charger 102 (i.e., in the charging mode).

In some embodiments, when operating in either the charging or measurement modes, processor 117 does not apply a neural stimulus to the neural pathway. Rather, processor 117 is configured to only measure non-evoked neural activity in measurement mode. That is, the processor 117 may be configured to detect electrical field signal parameters that are not produced or evoked by the implantable device 110 via the measurement of non-evoked neural activity. For example, the signal passing along the neural pathway may indicate that the patients bladder is full. A measurement of this signal may be taken during measurement mode, when charger 102 is not transmitting electromagnetic radiation to device 110.

The functionality of the previously described embodiments, in which processor 117 selectively transitions between operating in the charging mode and the measurement mode, is performed by the implantable device 110 as part of a “smart charging” operational routine. In some embodiments the implantable device 110 is configured to transition between various operational routines, including: the smart charging operational routine; a conventional feedback based operational routine, where the stimulus signal is altered based on the ECAP measurements without compensating for any charging activity (e.g., when the implantable device 110 operates without charger 102); and a static stimulus operational routine where a neural stimulus is applied to a neural pathway without any dynamic adjustment of the neural stimulus signal (irrespective of whether measurement of a corresponding neural compound action potential response evoked by the applied stimulus is performed). In some cases, the implantable device 110 may measure an electrical parameter associated with the tissue of the patient 108 whilst applying static stimulus. The implantable device 110 may cease the application of static stimulus, measure the electrical field parameter, and then resume the application of the static stimulus. In this case, the measured electrical field parameter is not used to adjust or alter the neural stimulus.

In some embodiments, the implantable device 110 is configured to detect the presence of the charger 102 within a charging proximity of the implantable device 110, and to automatically transition to the smart charging operational routine in response to the detection. That is, the implantable device 110 may transition between operational routines to optimize its function depending on the environment of its operation, and whether the implantable device 110 is receiving electromagnetic (EM) radiation 106 from an emitting device (i.e., either the charger 102, or another device).

For example, it may be desirable for the implantable device 110 to function in a static stimulus operational routine when the charger 102 is unavailable and when the implantable device 110 is subject to electromagnetic radiation 106 in the form of noise generated by another emitting device (e.g., an arc welder). In this situation, attempting to measure the ECAP response signal, such as for the purpose of performing feedback based control of the applied stimulus, may lead to a worse outcome than the static approach (i.e., where the inaccurate measurement of the evoked CAP signal results in maladjustment of the intensity of the induced stimuli).

In other situations, such as when the implantable device 110 does not receive electromagnetic radiation from the charger 102, or any other emitting device, an operational routine without stimulation and charging cycle functionality may be advantageous (e.g., since the measurement of the ECAP is not temporally restricted to occur within any particular period outside of a charging cycle).

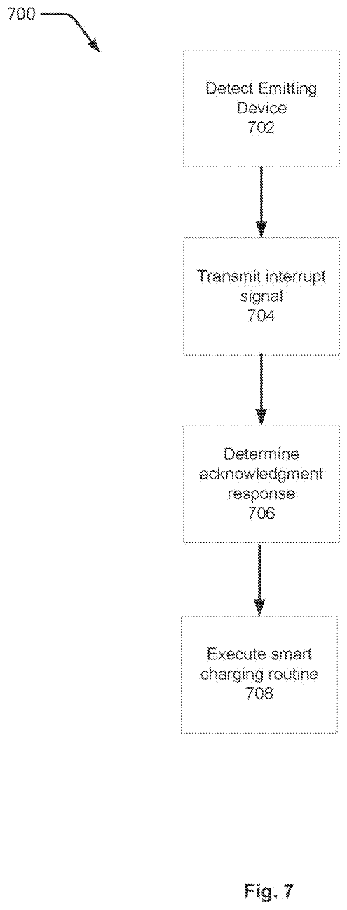

FIG. 7 illustrates a process 700 performed by the implantable device 110 for automatically transitioning to a smart charging operational routine on detection of the charger 102. At step 702, the implantable device 110 detects the presence of an emitting device within a charging distance of the implantable device 110. The charging distance is the maximum distance at which emitting device is capable of inducing a charging current in the implanted device 110, and therefore a potential in the measurement electrodes such as to affect the detection of the CAP signal (as described above). The charging distance is variable depending on the properties of the implantable and emitting devices.

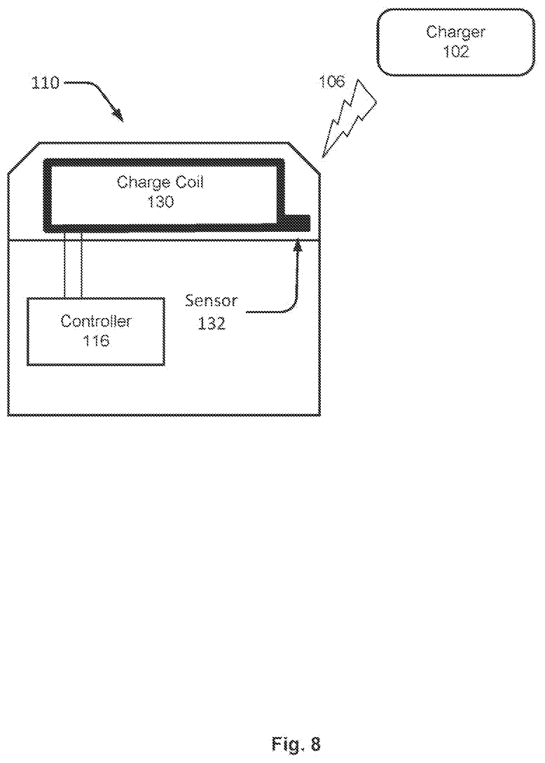

FIG. 8 illustrates an exemplary configuration of the implantable device 110 including: a charging coil 130 configured to convert electromagnetic radiation 106 received from charger 102 to a charging current in the implanted device 110; and a sensor 132 associated with the charging coil 130. In some embodiments, the charging coil 130 is formed from a Litz wire, or other type of multi-strand wire, with at least 100 strands of wire per cable. The sensor 132 is configured to detect the electromagnetic radiation 106, and therefore the presence of the charger 102 or other emitting device, within the charging distance. For example, the sensor 132 may be a component that senses the voltage and/or current induced by the EM radiation 106 from the charger 102.

In some embodiments, the sensor 132 is electrically connected to the coil 130 and is configured to measure the charge induced by EM radiation 106. In other embodiments, the sensor 132 may be an independent detection component physically located in the vicinity of the coil 130. In this case, the intensity of the EM radiation incident on the sensor 132 corresponds to that of the coil 130 for the purpose of enabling the sensor 132 to determine the proximity of the charger 102 to the device 110 . The sensor 132 is configured to output a detection signal based on the intensity of the incident EM radiation 106. The processor 117 determines a positive detection of the charger 102 in response to a value of the detection signal exceeding a predetermined detection threshold value.

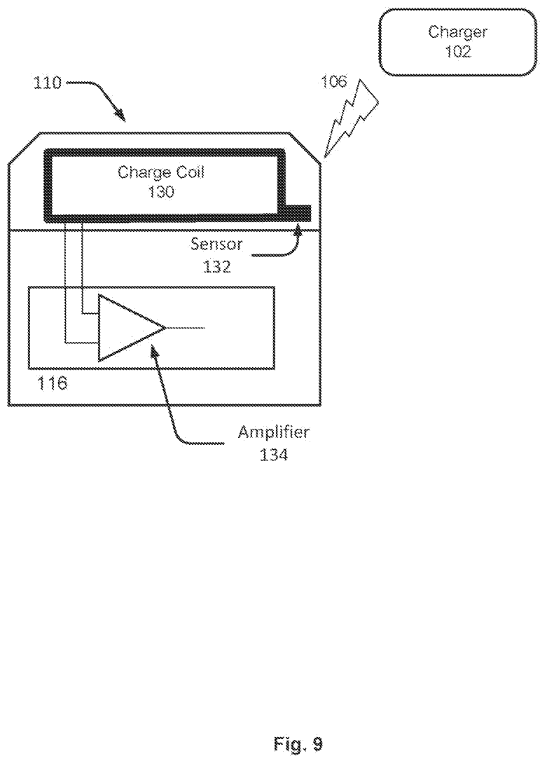

In another exemplary configuration, as shown by FIG. 9, the controller 116 of the implantable device 110 includes an amplifier 134 configured to sense noise resulting from the EM radiation 106. The amplifier is a differential amplifier, as described in U.S. Pat. No. 9,386,934 which is incorporated herein by reference. The amplifier 134 senses a noise associated with the electromagnetic radiation 106 received from the emitting device (i.e., the charger 102 as depicted in FIG. 8). The processor 117 receives an output noise signal from the amplifier 134 and detects the presence of the emitting device (e.g., charger 102) based at least in part on the noise signal. That is, the EM sensing amplifier 134 detects the proximity of the charger 102, or other emitting device, to the implantable device 110 through the noise resulting from the incident EM radiation 106 thereby providing an alternative to measuring the induced charge.

Referring back to FIG. 7, at steps 704 and 706 the implantable device 110 verifies whether the emitting device (detected in step 702) is the charger 102. Specifically, the implantable device 110 is configured to detect any emitting device acting as a source of EM radiation 106, such as for example via the sensor 132 described above. In some embodiments, verification of the charger 102 is based on the transmission of at least one interrupt signal to the emitting device, and the detection of a corresponding response of the emitting device that indicates whether the emitting device is the charger 102 or otherwise.

At step 704, the processor 117 transmits one or more interrupt signals to the emitting device. The form of the interrupt signal and its transmission may vary according to different embodiments of the device 110. For example, in some embodiments the processor 117 continuously transmits a single interrupt signal for a predetermined period of time. In other embodiments, the processor 117 transmits each interrupt signal as a short duration pulse signal over the predetermined period. The predetermined period during which the processor 117 transmits interrupt signal(s) is referred to as the interrupt period.

At step 706, the processor 117 is configured to detect a response of the emitting device to the one or more interrupt signals. The response that enables the processor 117 to positively verify the emitting device as the charger 102 is referred to as an acknowledgment response. In the described embodiments, the acknowledgment response involves the ceasing of the transmission of the EM radiation 106 by the emitting device. That is, positive verification of the emitting device as the charger 102 is achieved when the transmission of an interrupt signal causes the ceasing of the transmission EM radiation 106 by the emitting device (i.e., as an acknowledgment response). Processor 117 detects the ceasing of the transmission of the EM radiation 106, as a response to the interrupt signal of the implantable device 110, in the same manner as for the embodiments described above.

In some embodiments, the acknowledgment response also includes the recommencement of transmission of electromagnetic radiation 106 by the emitting device. For example, the acknowledgment response may involve the emitting device ceasing transmitting EM radiation 106, and then recommencing transmission according to a pre-determined duty cycle and predetermined frequency. Following the transmission of the one or more interrupt signals, the processor 117 detects the corresponding pattern associated with the transmission of EM radiation 106 according to the pre-determined duty cycle in which electrical energy transmission occurs intermittently with periodic repetition (as discussed above), and subsequently verifies the emitting device as the charger 102. In some embodiments, the processor 117 is configured to utilize a detection of the recommencement of transmission of EM radiation 106, following an initial ceasing of the transmission, within a single acknowledgment period to distinguish between the absence of any emitting device, and the presence of the charger 102 operating according to a predetermined duty cycle.

In some embodiments, the period in which the processor 117 determines the existence of the acknowledgment response, or otherwise, is referred to as the acknowledgment period. The acknowledgment period may commence following the interrupt period, such as for example when the interrupt signal is a single continuous signal. Alternatively, in other configurations the acknowledgment period may overlap with the interrupt period.

At step 708, in response to the processor 117 positively verifying the emitting device as the charging device 102, the processor 117 transitions the implantable device 110 from any other operational routine to the smart charging operational routine (i.e., such that the processor 117 operates according to the measurement and charging modes described herein).

In some embodiments, the verification of the emitting device as the charging device is performed without the transmission of interrupt signals by the implantable device 110. In some embodiments where the controller 116 includes the sensing amplifier 134, the processor 117 is configured to determine the noise as being associated with the charging device 102. That is, the processor 117 maintains a noise profile indicative of the EM noise characteristics produced by the charger 102 when located within the charging distance of the device 110.

For example, the noise profile may be in the form of a numerical or statistical model defined by one or more parameters of a noise signal feature space. The noise profile may be created by classification and/or model training operations performed on an external computer system, and by subsequently loading the output profile into the memory 118 of controller 116. The processor 117 is configured to compare feature values of the noise signal output by the amplifier 134 (i.e., the noise signature of the emitting device) to corresponding parameters of the noise profile to determine whether the noise signature is recognized as being of the charger 102 (i.e., as represented by the noise profile). Identification of the charger 102 via noise signature recognition is advantageous in that the processor 117 does not need to rely on the proper reception of interrupt signals by the emitting device, and need not wait for the emitting device to provide the acknowledgment response to achieve positive verification.

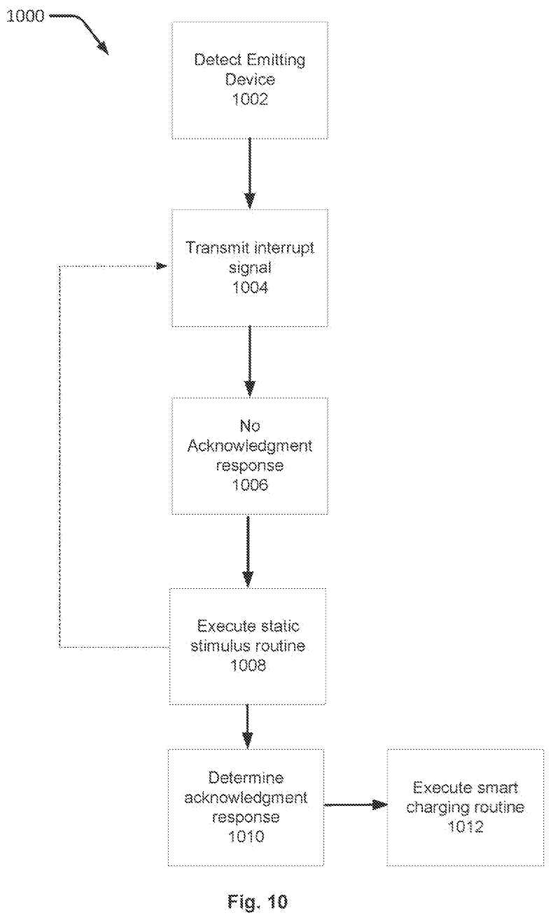

The implantable device 110 is also configured to automatically transition to other operational routines in response to determining that an emitting device is not the charger 102. For example, FIG. 10 illustrates a process 1000 performed by the implantable device 110 for automatically switching to an static stimulus signal based routine. At steps 1002 and 1004, the processor 117 detects the presence of an emitting device within a charging distance of the implantable device 110, and transmits one or more interrupt signals to the emitting device (as described above for steps 702 and 704).

At step 1006, the processor 117 attempts to detect a response of the emitting device to the one or more interrupt signals. That is, the processor 117 transmits the one or more interrupt signals to the emitting device during a first interrupt period, and then attempts to determine an acknowledgment response during a corresponding first acknowledgment period. In some embodiments, the first acknowledgment period is of a predetermined length (e.g., between 20 and 1000 ms) and may commence directly following the interrupt period. In the exemplary process of FIG. 10, the first acknowledgment period terminates without the processor 117 determining the existence of the acknowledgment response of the charger 102 (i.e., involving the ceasing of the transmission EM radiation 106 by the emitting device). That is, in this case the processor 117 detects an absence of the acknowledgment response of the emitting device within the first acknowledgment period.

At step 1008, in response to detecting the absence of the acknowledgment response within the first acknowledgment period, the processor 117 causes the implantable device 110 to transition, from any other operational routine, to the static stimulus operational routine where the stimulus signal is not modified in response to a measurement of the ECAP signal.

In some embodiments, when the implantable device 110 is in the static stimulus operational routine the processor 117 is prevented from measuring the ECAP signal associated with any application of a neural stimulus signal. That is, the processor 117 is configured to apply a neural stimulus to one or more neural pathways without a corresponding measurement of an ECAP resulting from the applied stimulus. That is, the processor 117 has determined that the emitting device is not the charger 102, and prevents the implantable device 110 from measuring ECAP signals due to the presence of the EM radiation 106. This prevents noise associated with the EM radiation from compromising the measurement and subsequent use of an evoked CAP signal (as may occur in a conventional closed loop control routine).

In some other embodiments, when the implantable device 110 is in the static stimulus operational routine the processor 117 may measure the ECAP signal associated with the application of a neural stimulus signal, but is prevented from altering the neural stimulus signal based on the ECAP measurement (i.e., due to the likelihood of the noise associated with the EM radiation compromising the measurement).

In some embodiments, the implantable device 110 is configured to maintain the static stimulus operational routine for a predetermined period. This is referred to as the back-off period. Following termination of the back-off period, the processor 117 may repeat the interrupt transmission and acknowledgment response cycle (as indicated by the dashed line in FIG. 10). That is, the processor 117 retransmits the one or more interrupt signals to the emitting device during a second interrupt period, and attempts to determine an acknowledgment response during a corresponding second acknowledgment period. If the processor 117 detects the acknowledgment response during the second acknowledgment period (i.e., at step 1010), then the emitting device is positively verified as the charger 102. In this case, the processor 117 causes the implantable device 110 to transition from the static stimulus operational routine to the smart charging operational routine (i.e., at step 1012) in the same manner as for the embodiments described above. Otherwise, the processor 117 initiates a further back-off period.

In some embodiments, the processor 117 is configured to repeat the interrupt transmission and acknowledgment response cycle until the charger 102 is detected, enabling the automatic transition to the smart charging operational routine, as described above. The processor 117 is configured to control the durations and commencement times of the respective interrupt, acknowledgment, and back-off periods of each cycle to facilitate the detection of the charger 102. For example, the acknowledgment period may be set to commence directly after the interrupt period, and with a duration set as equal to or greater than the duration of the charging cycle of the charger 102. This enables the detection of any ceasing of the transmission of electromagnetic radiation by the charger 102 within the corresponding acknowledgment period (since at least a portion of the second part of the charging cycle will fall within the acknowledgment period).

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

It should be understood that the techniques of the present disclosure might be implemented using a variety of technologies. For example, the methods described herein may be implemented by a series of computer executable instructions residing on a suitable computer readable medium. Suitable computer readable media may include volatile (e.g. RAM) and/or non-volatile (e.g. ROM, disk) memory, carrier waves and transmission media.

It should also be understood that, unless specifically stated otherwise as apparent from the following discussion, it is appreciated that throughout the description, discussions utilizing terms such as “estimating” or “processing” or “computing” or “calculating”, “optimizing” or “determining” or “displaying” or “maximising” or the like, refer to the action and processes of a computer system, or similar electronic computing device, that processes and transforms data represented as physical (electronic) quantities within the computer system's registers and memories into other data similarly represented as physical quantities within the computer system memories or registers or other such information storage, transmission or display devices.

The present embodiments are, therefore, to be considered in all respects as illustrative and not restrictive.

1. An implantable pulse generator device comprising a processor configured to:
(i) receive, in a charging mode, electromagnetic radiation from a charging device wherein the electromagnetic radiation transfers energy to the implantable device to charge an energy storage device;
(ii) measure, in a measurement mode, an electrical field parameter signal representing a neural response; and
(iii) selectively transition between the charging mode and the measurement mode, such that the implantable device does not receive electromagnetic radiation from the charging device during the measurement of the electrical field parameter signal.

2. The device of claim 1 wherein the processor is further configured to apply a neural stimulation signal to a neural pathway in at least one of: the charging mode; and the measurement mode.

3. The device of any of claims 1 to 2, wherein the processor is further configured to signal the charging device to cease transmission of the electromagnetic radiation before the measurement of the electrical field parameter signal.

4. The device of claim 3, wherein the signal to the charging device to cease transmission of the electromagnetic radiation is a reflected impedance indicative of a charged energy storage device.

5. The device of claim 4, wherein the reflected impedance is maintained for a stimulation-recording period during which a neural stimulus signal is applied and a corresponding electrical field parameter signal is measured.

6. The device of any of claims 1 to 5, wherein the signal to the charging device to cease transmission of the electromagnetic radiation is a wireless electromagnetic radio frequency signal.

7. The device of claim 6, wherein the radio frequency signal is within a medical implant communication service (MICS) band.

8. The device of any of claims 1 to 7, wherein the signal to the charging device to cease transmission of the electromagnetic radiation is maintained while in the measurement mode.

9. The device of any of claims 1 to 8, wherein the processor periodically operates in the measurement mode.

10. The device of any of claims 1 to 8, wherein the processor is configured to perform an adhoc transition to the measurement mode at random instances including one or more of: a time when the charger begins actively transferring electromagnetic radiation; and a time when the implantable device is directed to measure the electrical field parameter signal.

11. The device of any of claims 1 to 10, wherein the measurement of the electrical field parameter signal may include the measurement of at least one of: an ECAP; a non-evoked CAP;
a local field potential (LFP); a slow response; stimulus artefact; a physiological parameter; or a like electrical field parameter.

12. A method performed by a processor of an implantable pulse generator device to charge an energy storage device, the method comprising:
(i) receiving, in a charging mode, electromagnetic radiation from a charging device wherein the electromagnetic radiation transfers energy to the implantable device to charge an energy storage device;
(ii) measuring, in a measurement mode, an electrical field parameter signal representing a neural response; and
(iii) selectively transitioning between the charging mode and the measurement mode, such that the implantable device does not receive electromagnetic radiation from the charging device during the measurement of the electrical field parameter signal.

13. A system comprising:
an implantable pulse generator device comprising a processor configured to:
(i) receive, in a charging mode, electromagnetic radiation from a charging device wherein the electromagnetic radiation transfers energy to the implantable device to charge an energy storage device;
(ii) measure, in a measurement mode, an electrical field parameter signal representing a neural response; and
(iii) selectively transition between the charging mode and the measurement mode, such that the implantable device does not receive electromagnetic radiation from the charging device during the measurement of the electrical field parameter signal; and
a charger configured to transition from an active state to a standby state in response to receiving a signal from the implantable device, wherein in the active state the charger transmits electromagnetic radiation to the energy storage device and wherein in the standby state the charger does not transmit electromagnetic radiation to the energy storage device.

14. A non-transitory computer readable medium configured to store software instructions that when executed cause a processor to perform the method of claim 12.

15. A charger comprising a processor configured to transition from an active state to a standby state according to a predetermined duty cycle at a predetermined frequency, wherein in the active state the charger transmits electromagnetic radiation to an implantable pulse generator device and wherein in the standby state the charger does not transmit electromagnetic radiation to the implantable device.

16. The charger of claim 15, wherein the charger is further configured to:
receive an interrupt signal from the implantable device; and
in response to the received interrupt signal, perform at least one of: ceasing the transmission of electromagnetic radiation to the implantable device; and commencing the transmission of electromagnetic radiation to the implantable device.

17. The charger of any of claims 15 to 16, wherein the predetermined duty cycle is configurable based on a received configuration signal.

18. The charger of claim 17 wherein the configuration signal is received from the implantable device.

19. An implantable pulse generator device comprising a processor configured to selectively operate in one of a charging mode and a measurement mode, wherein the processor is further configured to:
detect electromagnetic radiation from a charging device wherein the electromagnetic radiation transfers energy to the implantable device to charge an energy storage device and wherein the electromagnetic radiation is transferred at a predetermined frequency and duty cycle to transfer energy during a first part of a charge cycle and to stop transferring energy during a second part of the charge cycle;
receive, in the charging mode, electromagnetic radiation from the charging device during the first part of the charge cycle;
apply a neural stimulus to a neural pathway; and
measure, in the measurement mode, an electrical field parameter signal during the second part of the charge cycle.

20. The device of claim 19, wherein the processor is further configured to:
determine the predetermined frequency and duty cycle from the electromagnetic radiation; and
perform stimulation cycles at the predetermined frequency to synchronise the stimulation cycles with the second part of the charge cycle, wherein each stimulation cycle comprises applying a neural stimulus to the neural pathway, and measuring, in the measurement mode, the electrical field parameter signal.

21. The implantable pulse generator device of any of claims 1 to 11, wherein the processor is further configured to:
detect the presence of an emitting device within a charging distance of the implantable device, the emitting device transmitting electromagnetic radiation to the implantable device;
verify whether the detected emitting device is the charging device; and
in response to a positive verification of the emitting device as the charging device, cause the implantable device to transition, from any other operational routine, to a smart charging operational routine in which the processor executes steps (i) to (iii).

22. The implantable pulse generator device of claim 21, wherein the processor detects the presence of the emitting device by processing a detection signal received from a sensor associated with a charging coil of the implantable device.

23. The implantable pulse generator device of claim 21, wherein the processor detects the presence of the emitting device by processing a detection signal received from an amplifier component of the implantable device,
wherein the amplifier component is configured to measure a noise signal associated with the electromagnetic radiation received from the emitting device.

24. The implantable pulse generator device of claim 23, wherein verifying the emitting device as the charging device includes processing the noise signal to recognize a noise signature associated with the charging device.

25. The implantable pulse generator device of any of claims 21 to 24, wherein the processor is further configured to: transmit one or more interrupt signals to the emitting device; and detect a response of the emitting device to the one or more interrupt signals, to verify whether the emitting device is the charging device.

26. The implantable pulse generator device of claim 25, wherein the response of the emitting device that enables the processor to positively verify the emitting device as the charging device is an acknowledgment response involving the ceasing of the transmission of the electromagnetic radiation by the emitting device.

27. The implantable pulse generator device of claim 26, wherein the processor is further configured to:
detect an absence of the acknowledgment response of the emitting device within a first pre-determined time period; and
in response to the absence of the acknowledgment response within the first pre-determined time period, cause the implantable device to transition, from any other operational routine, to a static stimulus operational routine in which the processor does not execute steps (i) to (iii), and instead executes the step of:
applying a neural stimulus without a corresponding measurement of an electrical field parameter signal.

28. The implantable pulse generator device of claim 27, wherein the processor is further configured to:
detect the presence of the acknowledgment response of the emitting device within a second pre-determined time period occurring after the first time period; and
in response to the detection of the acknowledgment response in the second time period, positively verify the emitting device as the charging device, to cause the implantable device to transition from the static stimulus operational routine to the smart charging operational routine.

29. The method of claim 12, further comprising:
detecting the presence of an emitting device within a charging distance of the implantable device, the emitting device transmitting electromagnetic radiation to the implantable device;
verifying whether the detected emitting device is the charging device; and
in response to a positive verification of the emitting device as the charging device, causing the implantable device to transition, from any other operational routine, to a smart charging operational routine in which the processor executes steps (i) to (iii).

30. The method of claim 29, further comprising: transmitting one or more interrupt signals to the emitting device; detecting a response of the emitting device to the one or more interrupt signals; and verifying, based on the response of the emitting device, whether the emitting device is the charging device.

31. The method of claim 30, further comprising:
detecting an absence of an acknowledgment response of the emitting device within a first pre-determined time period, wherein the acknowledgment response involves the ceasing of the transmission of the electromagnetic radiation by the emitting device; and
in response to the absence of the acknowledgment response within the first pre-determined time period, causing the implantable device to transition, from any other operational routine, to a static stimulus operational routine in which the processor does not execute steps (i) to (iii), and instead executes the step of:
applying a neural stimulus without a corresponding measurement of an electrical field parameter signal.

32. The method of claim 31, further comprising:
detecting the presence of the acknowledgment response of the emitting device within a second pre-determined time period occurring after the first time period; and
in response to the detection of the acknowledgment response in the second time period, positively verifying the emitting device as the charging device, to cause the implantable device to transition from the static stimulus operational routine to the smart charging operational routine.
